(19) United States
(12) Patent Application Publication
(10) Pub. No.: US 2002/0131990 A1
(43) Pub. Date: Sep. 19, 2002

(54) Pullulan free edible film compositions and methods of making the same

(76) Inventors: David G. Barkalow, Deerfield, IL (US); Albert H. Chapdelaine, Naperville, IL (US); Michael R. Dzija, LaGrange Park, IL (US)

Correspondence Address: Robert M. Barrett, Bell, Boyd & Lloyd, P.O. Box 1135, Chicago, IL, 60690-1135, US

(21) Appl. No.: 10/036,681

(22) Filed: Nov. 8, 2001

Related U.S. Application Data
(60) Provisional application No. 60/250,601, filed on Nov. 30, 2000.

(51) Int. Cl. A61K 9/14; A23L 1/212

(52) U.S. Cl. 424/439; 426/616

(57) ABSTRACT

Improved and inexpensive pullulan free edible film formulations and methods for making the same are provided. The edible pullulan free film compositions comprise effective amounts of at least one film forming agent; at least one bulk filler agent; at least one plasticizing agent; and optionally at least one thickening agent. Medicaments and other additive agents can also be incorporated into the edible films. The present invention further provides methods of delivering a medicament, treating halitosis, treating xerostomia, and treating plaque or gingivitis using the pullulan free edible film compositions.

### BACKGROUND OF THE INVENTION

The present invention generally relates to edible films. More specifically, the present invention relates to pullulan free edible film compositions and methods of making the same.

Oral cleansing and breath freshening may be difficult or inconvenient at times, depending on the nature of the breath freshening desired and the situation in which the breath freshening must occur. Brushing, flossing, cleaning your tongue and gargling using a variety of devices and compositions are common oral care practices well-suited for the privacy of one's home. However, such devices and compositions are less convenient to use away from the home where bathroom facilities might be scarce, unavailable or unsanitary. However, less obtrusive oral products have been developed. These include breath-freshening gums, lozenges, mouth sprays, and edible films.

Many thin edible films are made of pullulan, which may be used to deliver medicaments or other agents such as flavors and sweeteners to the oral cavity. However, pullulan is an expensive ingredient to use in the manufacture of such edible films because it has limited availability within the film formulation industry. Other edible materials such as modified starches and cellulosics have been employed as a replacement for pullulan within edible film compositions. Unfortunately, such materials typically lack one or more of pullulan's desirable film properties. Those properties include for example: rapid dissolution, flexibility, non-hygroscopity, clean mouth feel, clean flavor and ease of manufacture.

Therefore, there is a need within the film formulation industry for an improved pullulan free edible film composition and method of making the same comprised of film ingredients that have widespread availability, low cost, ease of manufacture, and which exhibit the desirable film properties found within pullulan-based edible films.

### SUMMARY OF THE INVENTION

The present invention provides improved pullulan free edible film compositions and methods of making the same. Moreover, the present invention further provides improved methods of delivering a medicament, treating halitosis, treating xerostomia and treating plaque or gingivitis utilizing the improved pullulan free edible film compositions.

To this end, the present invention provides a pullulan free edible film composition comprising an effective amount of at least one film forming agent; an effective amount of at least one bulk filler agent; and an effective amount of at least one plasticizing agent.

In an embodiment, the effective amount of the film forming agent ranges from approximately about 10% to about 90% dry weight of the composition.

In an embodiment, the film forming agent is chosen from the group consisting of: cellulose ethers; modified starches; natural gums; edible polymers; hydrocolloid flours; seaweed extracts; land plant extracts; derivatives thereof and combinations thereof.

In an embodiment, the effective amount of the bulk filler agent ranges from approximately about 10% to about 90% dry weight of the composition.

In an embodiment, the bulk filler agent is chosen from the group consisting of: magnesium carbonate; calcium carbonate; calcium phosphate; calcium sulfate; magnesium silicate; aluminum silicate; ground limestone; clay; talc; titanium dioxide; microcrystalline cellulose; cellulose polymers; derivatives thereof and combinations thereof.

In an embodiment, the effective amount of the plasticizing agent ranges up to approximately about 20% dry weight of the composition.

In an embodiment, the plasticizing agent is chosen from the group consisting of: glycerin; polyethylene glycol; propylene glycol; polyols; hydrogenated starch hydrolysates; corn syrups; derivatives thereof and combinations thereof.

In an embodiment, the pullulan free edible film composition further includes an effective amount of at least one thickening agent.

In an embodiment, the thickening agent is at least one cellulose ether.

In an embodiment, the pullulan free edible film composition further includes an effective amount of at least one medicament.

In an embodiment, the medicament is chosen from a group consisting of: pH control agents; oral care agents; breath freshening agents; pharmaceutical agents; nutraceutical agents; salivary stimulant agents; vitamins; minerals; derivatives thereof and combinations thereof.

In an embodiment, the pullulan free edible film composition further includes an effective amount of at least one additive agent.

In an embodiment, the additive agent is chosen from the group consisting of: surfactants; binding agents; coloring agents; sweeteners; flavorants; fragrances; emulsifiers; derivatives thereof and combinations thereof.

The present invention also provides a method of making a pullulan free edible film composition comprising the steps of providing an effective amount of at least one film forming agent and adding an effective amount of at least one bulk filler agent and an effective amount of at least one plasticizing agent with the film forming agent to produce the resultant pullulan free edible film composition.

In an embodiment, the method further includes the step of adding an effective amount of at least one thickening agent to form the pullulan free edible film composition.

Additionally, the present invention also provides a method of delivering a medicament comprising the steps of: providing a pullulan free edible film composition which includes an effective amount of at least medicament in the film composition to an individual, wherein the film composition further comprises an effective amount of at least one film forming agent; an effective amount of at least one bulk filler agent; and an effective amount of at least one plasticizing agent; and orally consuming the film composition by the individual to release the medicament into the oral cavity for absorption.

In an embodiment of the method, the medicament is a member chosen from a group consisting of pH control agents; oral care agents; breath freshening agents; pharmaceutical agents; nutraceutical agents; salivary stimulant agents; vitamins; minerals; derivatives thereof; and combinations thereof.

In an embodiment of the method, the method further includes the step of incorporating an effective amount of at least one thickening agent into the film composition.

Moreover, the present invention also provides a method of treating halitosis comprising the steps of: providing a pullulan free edible film composition which includes an effective amount of at least one breath freshening agent to an individual, wherein the film composition further comprises an effective amount of at least one film forming agent; an effective amount of at least one bulk filler agent; and an effective amount of at least one plasticizing agent; and orally consuming the film composition by the individual to release the breath freshening agent into the oral cavity to treat halitosis.

In an embodiment of the method, the method further comprises the step of incorporating an effective amount of at least one thickening agent into the film composition.

Additionally, the present invention further provides a method of treating xerostomia comprising the steps of: providing a pullulan free edible film composition which includes an effective amount of at least one salivary stimulant agent to an individual, wherein the film composition further comprises an effective amount of at least one film forming agent; an effective amount of at least one bulk filler agent; and an effective amount of at least one plasticizing agent; and orally consuming the film composition by the individual to release the salivary stimulant agent into the oral cavity to treat xerostomia.

In an embodiment of the method, the method further comprises the step of incorporating an effective amount of at least one thickening agent into the film composition.

In a still further embodiment, the present invention provides a method of treating plaque or gingivitis comprising the steps of: providing a pullulan free edible film composition which includes an effective amount of at least one oral care agent to an individual, wherein the film composition further comprises an effective amount of at least one film forming agent; and effective amount of at least one bulk filler agent; and an effective amount of at least one plasticizing agent; and orally consuming the film composition by the individual to release the oral care agent into the oral cavity to treat plaque or gingivitis.

In an embodiment of the method, the method further comprises the step of incorporating an effective amount of at least one thickening agent into the film composition.

Therefore, it is, an advantage of the present invention to provide an improved pullulan free edible film composition and a method for making the same which comprises ingredients that have widespread availability.

Another advantage of the present invention is to provide an improved pullulan free edible film composition and method for making the same which are inexpensive.

A further advantage of the present invention is to provide a pullulan free edible film composition that exhibits rapid dissolution, flexibility, non-hygroscopity, clean mouth feel, clean flavor and ease of manufacture.

A still further advantage of the present invention is to provide a chewing gum composition that includes a pullulan free edible film composition having widespread ingredient availability.

Additionally, another advantage of the present invention to provide a method of delivering a medicament to an individual utilizing a pullulan free edible film composition having widespread ingredient availability which is physiologically acceptable to the oral cavity, well adapted to adhere to oral surfaces and rapidly dissolves to release the medicament into the oral cavity for absorption via an open wound or mucous membrane.

Moreover, another advantage of the present invention is to provide an improved method of treating halitosis by delivering an effective amount of at least one breath freshening agent to the oral cavity of an individual using a pullulan free edible film composition having a film forming agent which entraps the breath freshening agent within the oral cavity to provide extended breath freshening efficacy.

Another advantage of the present invention is to provide an improved method of treating xerostomia by delivering an effective amount of at least one salivary stimulant agent to the oral cavity of an individual using a pullulan free edible film composition having a film forming agent which entraps the salivary stimulant agent within the oral cavity to provide extended salivary gland stimulation.

A further advantage of the present invention is to provide an improved method of treating plaque or gingivitis by delivering an effective amount of at least one oral care agent to the oral cavity of an individual using a pullulan free edible film composition having a film forming agent which entraps the oral care agent within the oral cavity to provide extended anti-plaque or anti-gingival effects.

Additional features and advantages of the present invention are described in and will apparent from, the detailed description of the presently preferred embodiments.

### DETAILED DESCRIPTION OF THE PRESENTLY PREFERRED EMBODIMENTS

Pursuant to the present invention, improved pullulan free edible film compositions and methods of making the same are provided. Additionally, improved methods of delivering a medicament and treatment of halitosis, xerostomia and plaque or gingivitis using the edible film compositions of the present invention are provided as well.

To this end, in an embodiment of the present invention a pullulan free edible film composition is provided. The composition includes an effective amount of at least one film forming agent; an effective amount of at least one bulk filler agent; and an effective amount of at least one plasticizing agent.

The film forming agent provides structure for the formed pullulan free edible film of the present invention. The effective amount of the film forming agent ranges from approximately about 10% to about 90%, more preferably 25% to about 75% dry weight of the film composition.

Film forming agents which can be used within the pullulan free edible film compositions of the present invention include, but are not limited to, cellulose ethers; modified starches; natural gums; edible polymers; hydrocolloid flours; seaweed extracts; land plant extracts; derivatives thereof; and combinations thereof.

Examples of cellulose ethers include, but are not limited to, methylcellulose; ethylcellulose; hydroxymethylcellulose; hydroxyethylcellulose; hydroxypropylmethylcellulose; carboxymethylcellulose; derivatives thereof and combinations thereof. Modified starch examples include, but are not limited to, acid and enzyme hydrolyzed corn and potato starches. Further, examples of natural gums include, but are not limited to, gum arabic; guar gum; locust bean gum; carageenan gum; acacia; karaya; ghatti; tragacanth agar; tamarind gum; xanthan gum; derivatives thereof; and combinations thereof.

Examples of edible polymers include, but are not limited to, microcrystalline cellulose; cellulose ethers; xanthan; derivatives thereof; and combinations thereof. Moreover, examples of hydrocolloid flour include, but are not limited to, guar gum; locust bean; microcrystalline cellulose; tara; derivatives thereof and combinations thereof.

Seaweed extract examples include, but are not limited to, alginates; carageenans; derivatives thereof; and combinations thereof. Land plant extract examples include, but are not limited to, konjac; pectin; arabinoglactan; derivatives thereof; and combinations thereof.

The preferred film forming agent of the present invention is carageenan.

It should be appreciated by those skilled in the art, that other edible water-soluble film forming agents which exhibit the desirable properties of the present invention and have widespread availability can also be used. The bulk filler agent of the present invention is added to the pullulan free edible film compositions to reduce the “slimy” texture of the compositions. The effective amount of the bulk filler agent ranges from approximately 10% to about 90%, more preferably 25% to about 75% by dry weight of the film composition.

Suitable bulk filler agents of the present invention include, but are not limited to, magnesium carbonate; calcium carbonate; calcium phosphate; calcium sulfate; magnesium silicate; aluminum silicate; ground lime stone; clay; talc; titanium dioxide; microcrystalline cellulose; cellulose polymers such as wood; derivatives thereof; and combinations thereof. The preferred bulk filler agent of the present invention is microcrystalline cellulose.

To improve flexibility and reduce brittleness of the pullulan free edible film compositions of the present invention, a softener, also known as a plasticizing agent, is used. The effective amount of the plasticizing agent of the present invention ranges from approximately 0% up to about 20%, more preferably 2% to about 8% dry weight of the film composition.

Suitable plasticizing agents of the present invention include, but are not limited to, polyols such as sorbitol; glycerin; polyethylene glycol; propylene glycol; hydrogenated starch hydrolysates; corn syrups; derivatives thereof; and combinations thereof. The preferred plasticizing agent of the present invention is polyethylene glycol.

To further enhance the structure of the film compositions of the present invention, an effective amount of at least one thickening agent may be used. Suitable thickening agents of the present invention include, but are not limited to, cellulose ethers. The preferred thickening agent of the present invention is hydroxyethyl cellulose.

In a preferred embodiment of the present invention, the pullulan free edible film composition comprises an effective amount of carageenan as a film forming agent; an effective amount of microcrystalline cellulose as a bulk filler agent; and an effective amount of polyethylene glycol as a plasticizing agent. Additionally, to enhance the structure of the formed film, an effective amount of hydroxyethyl cellulose as a thickening agent can be incorporated into the preferred embodiment.

The pullulan free edible film compositions of the present invention offer numerous advantages over comparable films within the prior art. The edible films of the present invention do not require the use of pullulan which significantly reduces their manufacturing cost. Because the components of the edible films of the present invention have widespread availability unlike pullulan-based films, the present invention offers an economic savings without loss of desirable properties.

Although not wanting to be bound to any particular theory, it is believed that the unique combination of film forming agents; bulk filler agents; plasticizing agents; and optionally thickening agents of the present invention produces a “stand alone” edible film composition which exhibits many of the same desirable properties found within more expensive pullulan-based films. It has been discovered that the pullulan free edible film compositions of the present invention exhibit rapid dissolution, non-hygroscopity, clean mouth feel, clean flavor and ease of manufacture like that of currently available pullulan-based films.

Moreover, the edible film compositions of the present invention overcome the deficiencies of other currently available pullulan free film compositions. The present invention provides a “stand alone” edible film which does not need the incorporation of expensive structural support ingredients. The present invention also exhibits more desirable edible film properties than other currently available pullulan free edible film compositions.

The film compositions of the present invention can also include an effective amount of at least one medicament and/or an effective amount of at least one additive agent. Medicaments which can be incorporated into the edible films of the present invention include, but are not limited to, pH control agents; oral care agents; breath freshening agents; pharmaceutical agents; nutraceutical agents; salivary stimulant agents; vitamins; minerals; derivatives thereof; and combinations thereof.

Examples of pH control agents include, but are not limited to, urea and orally acceptable buffers. Oral care agent examples include, but are not limited to, caries control agents such as phosphates and fluorides; anti-plaque/anti-gingivitis agents such as chlorohexidene, cetylpyridium chloride and triclosan; germ killing agents; and salivary stimulant agents such citric acid, lactic acid, malic acid, succinic acid, ascorbic acid, adipic acid, fumaric acid, tartaric acid and other edible food acids.

Breath freshening agents include, but are not limited to, zinc gluconate, citrus oils; fruit essences; peppermint oil; spearmint oil; other mint oils; clove oils; oil of wintergreen; anise; and menthol.

Pharmaceutical, nutraceutical, vitamin and mineral medicaments which may be incorporated within the edible film compositions of the present invention include, but are not limited to, those agents which are suitable for oral consumption and can be placed within film formulations.

Additives which can be incorporated into the edible film compositions of the present invention include, but are not limited to, surfactants; binding agents; coloring agents; sweeteners; flavorants; fragrances; emulsifiers; derivatives thereof; and combinations thereof.

Examples of surfactants and binding agents include any currently available edible surfactants and binding agents known within the film formulation arts. Coloring agent examples include, but are not limited to, food colors and dyes suitable for food, drug and cosmetic applications such as FD&C lakes and dyes. Sweeteners include, but are not limited to, natural sweeteners such as sucrose, dextrose, and maltose, and artificial sweeteners such as sucralose, aspartame, and salts of acesulfame.

A variety of flavorants can be used within edible films of the present invention too. Preferably, the flavorant comprises approximately 0.1 to about 20%, more preferably 10% to about 15% by dry weight of the edible film composition.

Suitable flavorants include, but are not limited to, essential oils and synthetic flavors or mixtures derived from plants and fruits. Artificial flavorants can also be used. Natural and artificial flavorants may be combined in any sensorially acceptable fashion.

Fragrance examples include, but are not limited to, any fragrance which is suitable for use within edible film formulations such as mint oils.

Emulsifiers which are suitable for use within the present invention include, but are not limited to, any emulsifier which is acceptable for use within edible film formulations such as partially hydrogenated vegetable oils.

It should be appreciated by those skilled in the art that other medicaments and additives not listed herein which are suitable for use within edible film formulations can be incorporated into the pullulan free film compositions of the present invention.

By way of example, and not limitation, the pullulan free edible film formulations set forth within Examples 1-12 below illustrate different embodiments of the present invention. It should be appreciated by those skilled in the art that the film formulations of the present invention can be produced in a variety of different manners that are appropriate for their intended purpose and market. Of course, many other formulations are possible and can be appreciated by a skilled technician once the fundamental concepts and principles of the present invention are grasped.

A wide range of changes and modifications to the embodiments of the present invention described below will be apparent to those skilled in the art. The following examples are not to be considered as imposing limitations on the present invention, but are merely included to illustrate various embodiments only.

Examples 1-12
(% dry weight)

| Ingredient | Example 1 | Example 2 | Example 3 | Example 4 |
|---|---|---|---|---|
| LustreClear* | 75.00 | 82.00 | 78.00 | 71.25 |
| Lecithin | 3.50 | 1.65 | 2.50 | 3.30 |
| Sorbitol | — | — | 2.50 | 3.75 |
| Glycerin | 4.50 | 4.00 | 2.00 | 3.50 |
| Flavor | 13.80 | 11.25 | 12.75 | 14.00 |
| Sucralose | 2.85 | 1.00 | 1.00 | — |
| Aspartame | — | — | 1.00 | 2.25 |
| Citric Acid | — | — | — | 1.00 |
| Menthol | 0.25 | — | — | 0.75 |
| Color | 0.10 | 0.10 | 0.25 | 0.20 |
| | 100.00 | 100.00 | 100.00 | 100.00 |
| Ingredient | Example 5 | Example 6 | Example 7 | Example 8 |
| Microcrystalline | 20.00 | 40.25 | 27.85 | — |
| Cellulose | | | | |
| Carageenan | 54.00 | 34.00 | 50.00 | 42.00 |
| Polyethylene | 2.00 | 8.00 | — | — |
| Glycol | | | | |
| Hydroxyethyl | — | 1.50 | 3.50 | — |
| Cellulose | | | | |
| Calcium | — | — | — | 39.00 |
| Carbonate | | | | |
| Lecithin | 3.00 | — | 2.00 | 2.00 |
| Sorbitol | — | 1.50 | 1.00 | — |
| Glycerin | 10.00 | — | 2.00 | 4.00 |
| Flavor | 9.00 | 11.75 | 13.00 | 10.50 |
| Sucralose | 1.85 | 1.50 | — | 2.00 |
| Aspartame | — | — | 0.50 | — |
| Citric Acid | — | 0.75 | — | — |
| Menthol | — | 0.65 | — | 0.25 |
| Color | 0.15 | 0.10 | 0.15 | 0.25 |
| | 100.00 | 100.00 | 100.00 | 100.00 |
| Ingredient | Example 9 | Example 10 | Example 11 | Example 12 |
| Microcrystalline | — | 19.00 | 44.00 | — |
| Cellulose | | | | |
| Guar Gum | — | — | — | 42.50 |
| Carageenan | — | 48.35 | 30.35 | — |
| Alginate | 37.50 | — | — | — |
| Polyethylene | — | 3.50 | 8.00 | — |
| Glycol | | | | |
| Hydroxyethyl | — | — | 3.00 | 6.50 |
| Cellulose | | | | |
| Calcium | 39.40 | 14.00 | — | 32.00 |
| Carbonate | | | | |
| Lecithin | — | 0.50 | 1.50 | — |
| Hydrogenated | 5.00 | — | — | — |
| Starch | | | | |
| Hydrolysates | | | | |
| Xylitol | 1.50 | — | — | 2.00 |
| Glycerin | — | — | 2.00 | 4.00 |
| Flavor | 13.90 | 11.00 | 8.00 | 10.00 |
| Sucralose | 1.00 | 2.50 | — | — |
| Acesulfame K | 1.00 | — | 3.00 | 2.00 |
| Chlorohexidene | 2.00 | — | — | — |
| Adipic Acid | | 1.00 | — | — |
| Menthol | 0.50 | — | — | 1.00 |
| Color | 0.10 | 0.15 | 0.15 | 0.00 |
| | 100.00 | 100.00 | 100.00 | 100.00 |

### EXAMPLES 1-4

To produce the film formulations of Examples 1-4, LustreClear® (a trademark of the FMC Corporation, intended for use as a clear coating for pharmaceutical tablets) is added to water while stirring. LustreClear® contains microcrystalline cellulose, carageenan, polyethylene glycol, hydroxyethyl cellulose and maltodextrin.

The mixture is then heated to 50° C. and other ingredients listed are added during stirring. Other ingredients may be added with the LustreClear® component before heating as well. While the mixture is warm, the mixture is poured onto a glass plate and drawn down to form a thin film with a 0.08″ blade. The film composition is then dried at 50° C. for approximately 15 minutes. The film will then thicken upon standing.

It was determined that the produced films of Examples 1-4 exhibited good flexibility, non-hygroscopity and ease of manufacture. Additionally, following formation of the films within Examples 1-4, a benchmark level sensory analysis was completed. It was determined that the films produced within Examples 1-4 exhibited good mouth feel, rapid dissolution, clean flavor and texture.

### EXAMPLES 5-12

To produce the film formulations of Examples 5-12, a film forming agent, bulk filler agent and plasticizing agent are added to water while stirring. The mixture is then heated to a temperature of 50° C. and other ingredients may be added while stirring the mixture. While the mixture is warm, the mixture is poured onto a glass plate and drawn down with an appropriately sized blade to form a thin film. The film is then dried at 50° C. for 15 minutes to thicken the film. It was determined that the produced films of Examples 5-12 exhibited good flexibility, non-hygroscopity and ease of manufacture.

Examples 1-12 illustrate that the edible pullulan free film formulations of the present invention utilize ingredients which have widespread ingredient availability. Moreover, those ingredients in such a unique combination exhibit desirable film properties which could only previously be produced within more expensive pullulan-based edible film formulations. However, because of the widespread availability of the ingredients of the present invention, a lower cost film exhibiting rapid dissolution, clean mouth feel, flexibility and ease of manufacture can now be produced.

In an embodiment, the present invention provides a method of making a pullulan free edible film composition as a “stand alone” film is provided. The method comprises the steps of providing an effective amount of at least one film forming agent; and adding an effective amount of at least one bulk filler agent and an effective amount of at least one plasticizing agent to the film forming agent to produce a pullulan free edible film composition.

The method may further comprises the step of adding an effective amount of at least one thickening agent to the edible film composition to enhance the structure of the film. Unlike currently available edible films, the method of the present invention produces a pullulan free edible film composition which is “stand alone.”

As a result, the pullulan free edible film compositions produced according to the method of the present invention do not require the use of additional expensive structural forming components such as pullulan and the like. Rather, the films are produced using ingredients that have widespread availability which lessens the cost of producing such films.

Further, when the ingredients of the films of the present invention are combined in a such a unique manner, desirable film properties can be achieved which until the present invention could only be accomplished by pullulan-based edible films. The method of the present invention offers a cost-effective alternative to pullulan-based films without loss of desirable film properties. In a still further embodiment of the present invention, a method of delivering a medicament is provided. The method comprises the steps of providing a pullulan free edible film composition which includes an effective amount of at least one medicament in the film composition to an individual, wherein the film composition further comprises an effective amount of at least one film forming agent; an effective amount of at least one bulk filler agent; and an effective amount of at least one plasticizing agent; and having the individual orally consume the film composition to release the medicament into the oral cavity for absorption.

Medicaments suitable for location within the pullulan free edible film composition include, but are not limited to, pH control agents; oral care agents; breath freshening agents; pharmaceutical agents; nutraceutical agents; salivary stimulant agents; vitamins; minerals; derivatives thereof and combinations thereof.

The method further comprises the step of incorporating an effective amount of at least one thickening agent into the film composition to enhance the structure of the film as a vehicle for delivery of the medicament. Again, although not wanting to be bound to any particular theory, it is believed that the edible film compositions of the present invention are more adapted to adhere to oral surfaces; more physiologically acceptable to the oral cavity; and more rapidly dissolve within the oral environment than currently available pullulan and pullulan free edible film compositions.

In doing so, it is believed that the pullulan free edible film compositions of the present invention more uniformly release the medicament into the oral cavity for absorption via an open wound or mucous membrane in a greater manner than could be previously achieved. Moreover, it is also believed that the film forming agent of the present invention entraps the medicament within the oral cavity for an extended period of time to prolong and enhance the effects of the medicament. In addition, by extending the contact time of the medicament within the oral cavity, the medicament is absorbed to a greater extent thereby increasing its bioavailability.

In another embodiment of the present invention, a method of treating halitosis is provided. Halitosis is often referred to as a conditions of malodorous breath. The method of treatment comprises the steps of providing a pullulan free edible film composition which includes an effective amount of at least one breath freshening agent to an individual, wherein the film composition further comprises an effective amount of at least one film forming agent; an effective amount of at least one bulk filler agent; and an effective amount of at least one plasticizing agent; and having the individual orally consume the film composition to release the breath freshening agent into the oral cavity to treat halitosis. The method may further comprise the step of incorporating an effective amount of at least one thickening agent into the film composition to enhance the structure of the film.

In a preferred embodiment of the method, the breath freshening agent is zinc gluconate. It is believed the halitosis treatment method of the present invention is more effective than currently available halitosis treatment modalities because the pullulan free edible film composition entraps the breath freshening agent within the oral cavity. By entrapping the breath freshening agent, the agent remains within the oral cavity for a longer period of time to provide extended breath freshening effects.

Thus, the method of treating halitosis of the present invention reduces the number of treatments an individual would require utilizing conventional halitosis aids. Because the breath freshening agent exists within the oral cavity via the method of the present invention, re-treatment with the breath freshening agent frequently is not required.

In a further embodiment of the present invention, a method of treating xerostomia is provided. Xerostomia is a medical condition in which an excessive dryness within the oral cavity occurs. A variety of medical conditions and pharmaceutical treatments cause xerostomia, requiring a need for a xerostomic treatment.

The method of treating xerostomia of the present invention comprises the steps of providing a pullulan free edible film composition which includes an effective amount of at least one salivary stimulant agent to an individual, wherein the film composition comprises an effective amount of at least one film forming agent; an effective amount of at least one bulk filler agent; and an effective amount of at least one plasticizing agent; and having the individual orally consume the film composition to release the salivary stimulant agent into the oral cavity to treat xerostomia. Additionally, the method further comprises the step of incorporating an effective amount of at least one thickening agent into the film composition to enhance the structure of the film.

By entrapping the salivary stimulant agent within the oral cavity via the pullulan free edible film composition of the present invention, extended salivary gland stimulation can be achieved to a greater extent than could be previously accomplished with currently available xerostomic aids. Additionally, because the salivary stimulant agent is kept within the oral cavity for an extended period of time, salivary gland stimulation occurs over a longer period of time which decreases the need for frequent re-treatments.

Moreover, the method of treating xerostomia of the present invention also provides the additional advantage of allowing a xerostomic patient the ability to discreetly treat such a condition. The patient merely keeps the edible film or chewing gum composition of the present invention in a conventional storage mechanism such as a wrapper, and then conspicuously place the film or gum within the mouth when needed to provide salivary relief.

In a still further embodiment of the present invention, a method of treating plaque or gingivitis is provided. The method comprises the steps of providing a pullulan free edible film composition which includes an effective amount of at least one oral care agent to an individual, wherein the film composition further comprises an effective amount of at least one film forming agent; an effective amount of at least one bulk filler agent; and an effective amount of at least one plasticizing agent; and having the individual orally consume the film composition to release the oral care agent into the oral cavity to treat plaque or gingivitis. The method may further comprise the step of incorporating an effective amount of at least one thickening agent into the film composition to enhance the structure of the film.

Oral care agents which are suitable for use within the method include, but are not limited to, pH control agents such as urea and buffers; tartar or caries control agents such as phosphates and fluorides; anti-plaque/anti-gingival agents such as chlorohexidene, cetylpyridium chloride and triclosan; and germ killing combinations like that found within the brand name product, Listerine®.

The method provides a manner of entrapping the oral care agent within the oral cavity to extend the anti-plaque and/or anti-gingival effects of the agent. In doing so, the method of the present invention enhances the anti-cavity effects of these agents by keeping them within the oral cavity for a longer period of time than currently available edible film compositions. As a result, this method of the present invention allows individuals to achieve increased levels of dental care than could be previously done.

It should be understood that various changes and modifications of the presently preferred embodiments described herein will be apparent to those skilled in the art. Such changes and modifications can be made without departing from the spirit and scope of the present invention without diminishing its intended advantages. It is therefore intended that such changes and modifications be covered by the appended claims.

We claim:

1. A pullulan free edible film composition comprising:
an effective amount of at least one film forming agent;
an effective amount of at least one bulk filler agent; and
an effective amount of at least one plasticizing agent.

2. The composition of claim 1, wherein the effective amount of the film forming agent ranges from approximately about 10% to about 90% dry weight of the composition.

3. The composition of claim 2, wherein the film forming agent is a member selected from the group consisting of a cellulose ether, a modified starch, a natural gum, an edible polymer, a hydrocolloid flour, a seaweed extract, a land plant extract, derivatives thereof and combinations thereof.

4. The composition of claim 1, wherein the effective amount of the bulk filler agent ranges from approximately about 10% to about 90% dry weight of the composition.

5. The composition of claim 4, wherein the bulk filler agent is a member selected from the group consisting of magnesium carbonate, calcium carbonate, calcium phosphate, calcium sulfate, magnesium silicate, aluminum silicate, ground limestone, clay, talc, titanium dioxide, microcrystalline cellulose, cellulose polymers, derivatives thereof and combinations thereof.

6. The composition of claim 1, wherein the effective amount of the plasticizing agent ranges up to approximately about 20% dry weight of the composition.

7. The composition of claim 6, wherein the plasticizing agent is a member selected from the group consisting of a glycerin, a polyethylene glycol, a propylene glycol, a polyol, a hydrogenated starch hydrolysate, a corn syrup, derivatives thereof and combinations thereof.

8. The composition of claim 1, wherein the composition further includes an effective amount of at least one thickening agent.

9. The composition of claim 8, wherein the thickening agent is at least one cellulose ether.

10. The composition of claim 1, wherein the composition further includes an effective amount of at least one medicament.

11. The composition of claim 10, wherein the medicament is a member selected from the group consisting of a pH control agent, an oral care agent, a breath freshening agent, a pharmaceutical agent, a nutraceutical agent, a salivary stimulant agent, a vitamin, a mineral, derivatives thereof and combinations thereof.

12. The composition of claim 1, wherein the composition further includes an effective amount of at least one additive agent.

13. The composition of claim 12, wherein the additive agent is a member selected from the group consisting of a surfactant, a binding agent, a coloring agent, a sweetener, a flavorant, a fragrance, an emulsifier, derivatives thereof and combinations thereof.

14. A method of making a pullulan free edible film composition comprising the steps of:
providing an effective amount of at least one film forming agent; and
adding an effective amount of at least one bulk filler agent and an effective amount of at least one plasticizing agent with the film forming agent to produce a pullulan free edible film composition.

15. The method of claim 18, wherein the method further includes the step of adding an effective amount of at least one thickening agent to form the pullulan free edible film composition.

16. A method of delivering a medicament comprising the steps of:
providing a pullulan free edible film composition which includes an effective amount of at least one medicament in the film composition to an individual, wherein the film composition further comprises:
an effective amount of at least one film forming agent;
an effective amount of at least one bulk filler agent; and
an effective amount of at least one plasticizing agent; and
orally consuming the film composition by the individual to release the medicament into the oral cavity.

17. The method of claim 20, wherein the medicament is a member selected from the group consisting of a pH control agent, an oral care agent, a breath freshening agent, a pharmaceutical agent, a nutraceutical agent, a salivary stimulant agent, a vitamin, a mineral, derivatives thereof and combinations thereof.

18. The method of claim 20, wherein the method further includes the step of incorporating an effective amount of at least one thickening agent into the film composition.

19. A method of treating halitosis comprising the steps of:
providing a pullulan free edible film composition which includes an effective amount of at least one breath freshening agent to an individual, wherein the film composition further comprises:
an effective amount of at least one film forming agent;
an effective amount of at least one bulk filler agent; and
an effective amount of at least one plasticizing agent; and
orally consuming the film composition by the individual to release the breath freshening agent into the oral cavity to treat halitosis.

20. The method of claim 23, wherein the method further comprises the step of incorporating an effective amount of at least one thickening agent into the film composition.

21. A method of treating xerostomia comprising the steps of:
providing a pullulan free edible film composition which includes an effective amount of at least one salivary stimulant agent to an individual, wherein the film composition further comprises:
an effective amount of at least one film forming agent;
an effective amount of at least one bulk filler agent; and
an effective amount of at least one plasticizing agent; and
orally consuming the film composition by the individual to release the salivary stimulant agent into the oral cavity to treat xerostomia.

22. The method of claim 25, wherein the method further comprises the step of incorporating an effective amount of at least one thickening agent into the film composition.

23. A method of treating plaque or gingivitis comprising the steps of:
providing a pullulan free edible film composition which includes an effective amount of at least one oral care agent to an individual, wherein the film composition further comprises:
an effective amount of at least one film forming agent;
an effective amount of at least one bulk filler agent;
an effective amount of at least one plasticizing agent; and
orally consuming the film composition by the individual to release the oral care agent into the oral cavity to treat plaque or gingivitis.

24. The method of claim 27, wherein the method further comprises the step of incorporating an effective amount of at least one thickening agent into the film composition.
